(19) United States
(12) Patent Application Publication
(10) Pub. No.: US 2023/0201538 A1
(43) Pub. Date: Jun. 29, 2023

(54) Assemblies and Methods of Introducers and Rapidly Insertable Central Catheters

(71) Applicant: Bard Access Systems, Inc., Salt Lake City, UT (US)

(72) Inventors: Glade H. Howell, Draper, UT (US); Daniel B. Blanchard, Bountiful, UT (US); Eric W. Lindekugel, Salt Lake City, UT (US); Kyle G. Thornley, Farmington, UT (US)

(21) Appl. No.: 18/087,676

(22) Filed: Dec. 22, 2022

Related U.S. Application Data
(60) Provisional application No. 63/293,544, filed on Dec. 23, 2021.

(51) Int. Cl. A61M 25/09; A61M 25/06

(52) U.S. Cl. CPC A61M 25/09; A61M 25/0606; A61M 25/0668

(57) ABSTRACT

An introducer assembly can include an introducer and an access guidewire disposed therein. An introducer needle of the introducer can include a needle shaft having a needle slot extending from a proximal portion of the needle shaft through a distal needle tip. An introducer sheath of the introducer can include a sheath body over the needle shaft sealing the needle slot thereunder but for a sheath-body opening in a proximal portion thereof. The access guidewire can include a distal-end portion disposed in the introducer having an outer diameter greater than a proximal-end portion of the needle slot such that the proximal-end portion of the needle slot catches the distal-end portion of the access guidewire preventing proximal withdrawal of the access guidewire from the needle shaft. A method can include inserting a rapidly insertable central catheter into a blood-vessel lumen of a patient with the introducer assembly.

### PRIORITY

This application claims the benefit of priority to U.S. Provisional Application No. 63/293,544, filed Dec. 23, 2021, which is incorporated by reference in its entirety into this application.

### BACKGROUND

Central venous catheter (“CVCs”) are commonly introduced into patients and advanced through their vasculatures by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma. In addition, there is a relatively high potential for touch contamination due to the number of medical devices that need to be interchanged during the number of steps of the Seldinger technique. As such, there is a need to reduce the number of steps and medical devices involved in introducing a catheter such as a CVC into a patient and advancing the catheter through a vasculature thereof.

Disclosed herein are assemblies and methods of introducers and rapidly insertable central catheters (“RICCs”) that address the foregoing.

### SUMMARY

Disclosed herein is an introducer assembly including, in some embodiments, an introducer and an access guidewire disposed in the introducer. The introducer includes an introducer needle and a splittable introducer sheath over the introducer needle. The introducer needle includes a needle shaft having a longitudinal needle slot extending from a proximal portion of the needle shaft through a distal needle tip. The introducer sheath includes a sheath body over the needle shaft sealing the needle slot thereunder but for a sheath-body opening in a proximal portion of the sheath body. The access guidewire includes a distal-end portion disposed in the introducer. The distal-end portion of the access guidewire has an outer diameter greater than at least a proximal-end portion of the needle slot such that the proximal-end portion of the needle slot catches the distal-end portion of the access guidewire preventing proximal withdrawal of the access guidewire from the needle shaft.

In some embodiments, a proximal portion of the access guidewire has an outer diameter commensurate with the outer diameter of the distal-end portion of the access guidewire. In addition, an intermediary portion of the access guidewire between the proximal portion of the access guidewire and the distal-end portion of the access guidewire has an outer diameter less than that of the proximal portion of the access guidewire and the distal-end portion of the access guidewire.

In some embodiments, a proximal portion of the distal-end portion of the access guidewire includes a rigid tubular fitting thereover, thereby preventing the proximal portion of the distal-end portion of the access guidewire from bending. By preventing the proximal portion of the distal-end portion of the access guidewire from bending, the distal-end portion of the access guidewire is directed to engage the proximal-end portion of the needle slot over a remainder of the needle slot.

In some embodiments, the proximal-end portion of the needle slot has a same width as a remainder of the needle slot.

In some embodiments, the proximal-end portion of the needle slot is narrower than a remainder of the needle slot.

In some embodiments, the sheath-body opening extends over the proximal-end portion of the needle slot without substantially extending over the remainder of the needle slot such that the sheath body prevents the proximal withdrawal of the access guidewire from the needle shaft by interfering with the distal-end portion of the access guidewire.

In some embodiments, the introducer sheath further includes a splittable sheath hub around the proximal portion of the sheath body.

In some embodiments, the sheath hub extends over the remainder of the needle slot exposed by the sheath-body opening such that the sheath hub prevents the proximal withdrawal of the access guidewire from the needle shaft by interfering with the distal-end portion of the access guidewire.

In some embodiments, the sheath hub includes a port over the remainder of the needle slot exposed by the sheath-body opening. The port is configured to exclusively direct the access guidewire into the proximal-end portion of the needle slot, thereby preventing the proximal withdrawal of the access guidewire from the needle shaft.

In some embodiments, the introducer needle further includes a needle hub around a proximal-end portion of the needle shaft.

In some embodiments, the introducer assembly further includes a syringe. The syringe includes a tapered male syringe tip extending from a distal portion of the syringe. The syringe tip configured to insert into a tapered female needle-hub connector in a proximal portion of the needle hub.

Also disclosed herein is a RICC insertion assembly including, in some embodiments, a RICC, an introducer, and an access guidewire disposed in both the RICC and the introducer. The introducer includes an introducer needle and a splittable introducer sheath over the introducer needle. The introducer needle includes a needle shaft having a longitudinal needle slot extending from a proximal portion of the needle shaft through a distal needle tip. The introducer sheath includes a sheath body over the needle shaft sealing the needle slot thereunder but for a sheath-body opening in a proximal portion of the sheath body. The access guidewire includes a proximal portion disposed in the RICC and a distal-end portion disposed in the introducer. The distal-end portion of the access guidewire has an outer diameter greater than at least a proximal-end portion of the needle slot such that the proximal-end portion of the needle slot catches the distal-end portion of the access guidewire preventing proximal withdrawal of the access guidewire from the needle shaft.

In some embodiments, a proximal portion of the access guidewire has an outer diameter commensurate with the outer diameter of the distal-end portion of the access guidewire. In addition, an intermediary portion of the access guidewire between the proximal portion of the access guidewire and the distal-end portion of the access guidewire has an outer diameter less than that of the proximal portion of the access guidewire and the distal-end portion of the access guidewire.

In some embodiments, a proximal portion of the distal-end portion of the access guidewire includes a rigid tubular fitting thereover, thereby preventing the proximal portion of the distal-end portion of the access guidewire from bending. By preventing the proximal portion of the distal-end portion of the access guidewire from bending, the distal-end portion of the access guidewire is directed to engage the proximal-end portion of the needle slot over a remainder of the needle slot.

In some embodiments, the proximal-end portion of the needle slot has a same width as a remainder of the needle slot.

In some embodiments, the proximal-end portion of the needle slot is narrower than a remainder of the needle slot.

In some embodiments, the sheath-body opening extends over the proximal-end portion of the needle slot without substantially extending over the remainder of the needle slot such that the sheath body prevents the proximal withdrawal of the access guidewire from the needle shaft by interfering with the distal-end portion of the access guidewire.

In some embodiments, the introducer sheath further includes a splittable sheath hub around the proximal portion of the sheath body.

In some embodiments, the sheath hub extends over the remainder of the needle slot exposed by the sheath-body opening such that the sheath hub prevents the proximal withdrawal of the access guidewire from the needle shaft by interfering with the distal-end portion of the access guidewire.

In some embodiments, the sheath hub includes a port over the remainder of the needle slot exposed by the sheath-body opening. The port is configured to exclusively direct the access guidewire into the proximal-end portion of the needle slot, thereby preventing the proximal withdrawal of the access guidewire from the needle shaft.

In some embodiments, the introducer needle further includes a needle hub around a proximal-end portion of the needle shaft.

In some embodiments, the RICC insertion assembly further includes a syringe. The syringe includes a tapered male syringe tip extending from a distal portion of the syringe. The syringe tip configured to insert into a tapered female needle-hub connector in a proximal portion of the needle hub.

Also disclosed herein is a method for inserting a RICC into a blood-vessel lumen of a patient. The method includes, in some embodiments, an insertion assembly-acquiring step, a needle tract-establishing step, an access guidewire-advancing step, an introducer needle-withdrawing step, an introducer sheath-splitting step, and a RICC-advancing step. The insertion assembly-acquiring step includes acquiring a RICC insertion assembly including the RICC, an introducer, and an access guidewire disposed in both the RICC and the introducer in a substantially ready-to-operate state of the RICC insertion assembly. The introducer includes an introducer needle and a splittable introducer sheath over the introducer needle. The introducer needle includes a needle shaft having a longitudinal needle slot extending from a proximal portion of the needle shaft through a distal needle tip. The introducer sheath includes a sheath body over the needle shaft sealing the needle slot thereunder but for a sheath-body opening in a proximal portion of the sheath body. The access guidewire includes a proximal portion disposed in the RICC and a distal-end portion disposed in the introducer.

The distal-end portion of the access guidewire has an outer diameter greater than at least a proximal-end portion of the needle slot such that the proximal-end portion of the needle slot catches the distal-end portion of the access guidewire preventing proximal withdrawal of the access guidewire from the needle shaft. The needle tract-establishing step includes establishing a needle tract from an area of skin to the blood-vessel lumen with the introducer. The access guidewire-advancing step includes advancing a distal end of the access guidewire from its initial location in the introducer proximal of the needle tip into the blood-vessel lumen. The introducer needle-withdrawing step includes withdrawing the introducer needle from the introducer sheath leaving both the introducer sheath and the access guidewire in place in the blood-vessel lumen. The needle slot allows the removing of the introducer needle from the introducer sheath while the access guidewire remains in place in the blood-vessel lumen. The introducer sheath-splitting step includes splitting the introducer sheath away from the access guidewire to remove split portions of the introducer sheath from the blood-vessel lumen. The RICC-advancing step includes advancing a catheter tube of the RICC over the access guidewire and into the blood-vessel lumen, thereby inserting the RICC into the blood-vessel lumen.

In some embodiments, the method further includes a blood-aspirating step. The blood-aspirating step includes aspirating blood with a syringe coupled to the introducer needle for confirmation the needle tract extends into the blood-vessel lumen before withdrawing the introducer needle from the introducer sheath in the introducer needle-withdrawing step.

In some embodiments, the method further an access guidewire-withdrawing step. The access guidewire-withdrawing step includes withdrawing the access guidewire leaving the catheter tube in place in the blood-vessel lumen.

In some embodiments, the method further includes a maneuver guidewire-advancing step, an additional RICC-advancing step, and a maneuver guidewire-withdrawing step. The maneuver guidewire-advancing step includes advancing a maneuver guidewire into the blood-vessel lumen by way of a primary lumen of the RICC. The other RICC-advancing step includes advancing a distal portion of the catheter tube farther into the blood-vessel lumen over the maneuver guidewire to a lower ⅓ of a superior vena cava (“SVC”) of a heart of the patient. The maneuver guidewire-withdrawing step includes withdrawing the maneuver guidewire leaving the catheter tube in place in the lower ⅓ of the SVC.

These and other features of the concepts provided herein will become more apparent to those of skill in the art in view of the accompanying drawings and following description, which describe particular embodiments of such concepts in greater detail.

### DRAWINGS

FIG. 1 illustrates a RICC in accordance with some embodiments.

FIG. 2 illustrates a distal portion of a catheter tube of the RICC in accordance with some embodiments.

FIG. 3 illustrates a first transverse cross section of the distal portion of the catheter tube in accordance with some embodiments.

FIG. 4 illustrates a second transverse cross section of the distal portion of the catheter tube in accordance with some embodiments.

FIG. 5 illustrates a longitudinal cross section of the distal portion of the catheter tube in accordance with some embodiments.

FIG. 6 illustrates a first view of an introducer assembly including a syringe for inserting the RICC into a patient in accordance with some embodiments.

FIG. 7 illustrates a second view of the introducer assembly including the syringe in accordance with some embodiments.

FIG. 8 illustrates a third view of the introducer assembly including the syringe in accordance with some embodiments.

FIG. 9 illustrates a first view of an introducer including an introducer needle and a splittable introducer sheath in accordance with some embodiments.

FIG. 10 illustrates a second view of the introducer in accordance with some embodiments.

FIG. 11 illustrates a distal portion of the introducer in accordance with some embodiments.

FIG. 12 illustrates the introducer without a sheath hub of the introducer sheath for expository expediency.

FIG. 13 illustrates the introducer needle including a needle shaft and a needle hub in accordance with some embodiments.

FIG. 14 illustrates the introducer sheath including a sheath body and the sheath hub in accordance with some embodiments.

FIG. 15 illustrates a longitudinal cross section of the distal portion of the introducer assembly in accordance with some embodiments.

FIG. 16 illustrates a detailed view of a distal portion of the introducer assembly in accordance with some embodiments, wherein the introducer sheath is removed from the introducer needle for expository expediency.

FIG. 17 illustrates a detailed view of the distal portion of the introducer assembly in accordance with some other embodiments, wherein the introducer sheath is removed from the introducer needle for expository expediency.

FIG. 18 illustrates a detailed view of the distal portion of the introducer assembly in accordance with yet some other embodiments, wherein the introducer sheath is removed from the introducer needle for expository expediency.

FIG. 19 illustrates a longitudinal cross section of the distal portion of the introducer assembly of FIG. 18, wherein the introducer sheath is removed from the introducer needle for expository expediency.

FIG. 20 illustrates a detailed view of the distal portion of the introducer assembly of FIG. 21 with the introducer sheath.

FIG. 21 illustrates a longitudinal cross section of the distal portion of the introducer assembly of FIG. 18 with a rigid tubular fitting over an access guidewire in accordance with some embodiments.

FIG. 22 illustrates a longitudinal cross section of the distal portion of the introducer assembly of FIG. 18 with the sheath hub of the introducer sheath partially blocking a needle slot of the needle shaft in accordance with some embodiments.

FIG. 23 illustrates a RICC insertion assembly in accordance with some embodiments.

FIG. 24 illustrates a first view of a distal portion of the RICC insertion assembly in accordance with some embodiments.

FIG. 25 illustrates a second view of the distal portion of the RICC insertion assembly in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above, while the Seldinger technique is effective, the number of steps are time consuming, handling the number of medical devices is awkward, and both of the foregoing can lead to patient trauma. In addition, there is a relatively high potential for touch contamination due to the number of medical devices that need to be interchanged during the number of steps of the Seldinger technique. As such, there is a need to reduce the number of steps and medical devices involved in introducing a catheter such as a CVC into a patient and advancing the catheter through a vasculature thereof.

Disclosed herein are assemblies and methods of introducers and rapidly insertable central catheters that address the foregoing. For example, an introducer assembly can include an introducer and an access guidewire disposed in the introducer. An introducer needle of the introducer can include a needle shaft having a needle slot extending from a proximal portion of the needle shaft through a distal needle tip. An introducer sheath of the introducer can include a sheath body over the needle shaft sealing the needle slot thereunder but for a sheath-body opening in a proximal portion thereof. The access guidewire can include a distal-end portion disposed in the introducer. The distal-end portion of the access guidewire can have an outer diameter greater than a proximal-end portion of the needle slot such that the proximal-end portion of the needle slot catches the distal-end portion of the access guidewire preventing proximal withdrawal of the access guidewire from the needle shaft. These and other features of the assemblies and methods provided herein will become more apparent to those of skill in the art in view of the accompanying drawings and following description, which describe particular embodiments of such assemblies and methods in greater detail.

### RICCs

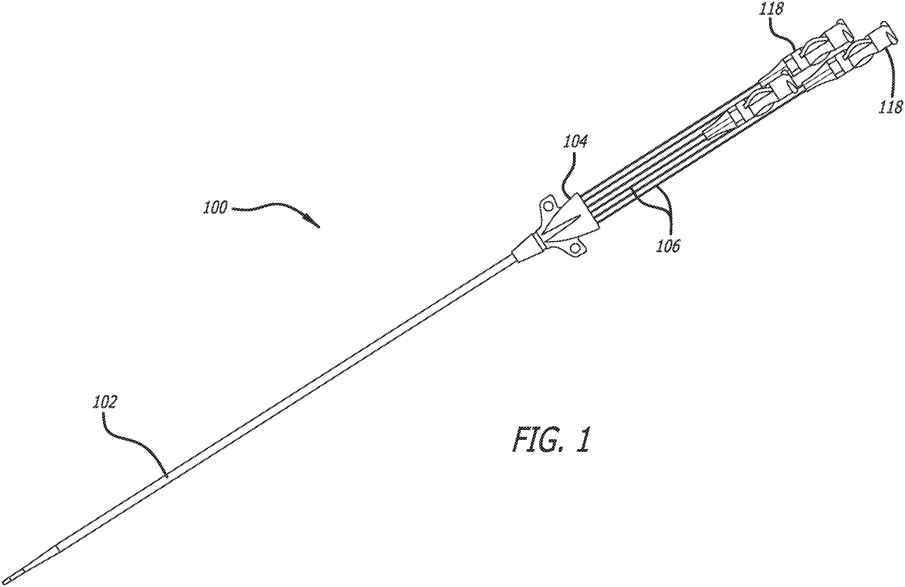

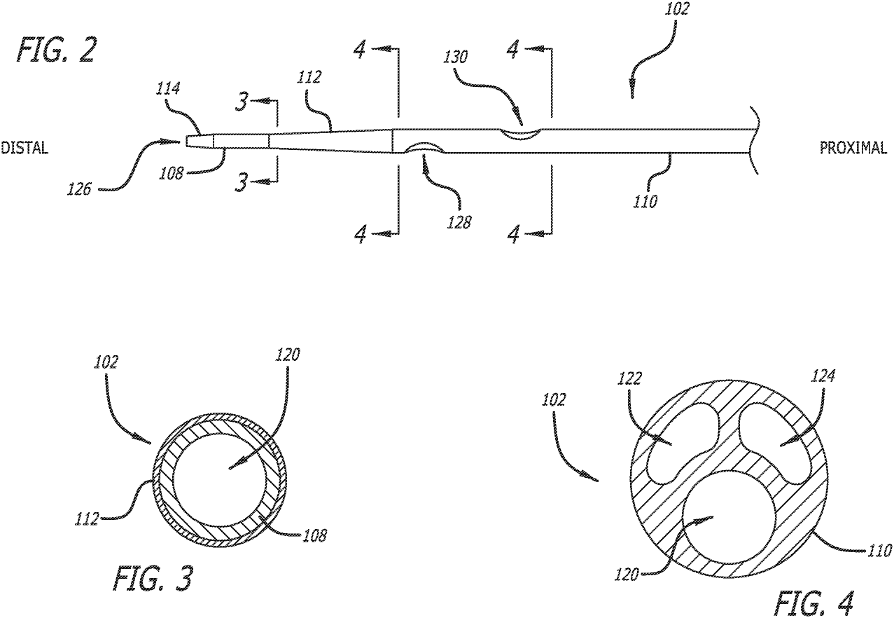

FIG. 1 illustrates a RICC 100 in accordance with some embodiments. FIG. 2 illustrates a distal portion of a catheter tube 102 of the RICC 100 in accordance with some embodiments. FIGS. 3-5 illustrate different cross sections of the distal portion of the catheter tube 102 in accordance with some embodiments.

As shown, the RICC 100 includes the catheter tube 102, a catheter hub 104, and one or more extension legs 106.

The catheter tube 102 includes a first section 108 in the distal portion of the catheter tube 102, a second section 110 in the distal portion of the catheter tube 102 proximal of the first section 108, and a tapered junction 112 between the first and second sections 108 and 110 of the catheter tube 102.

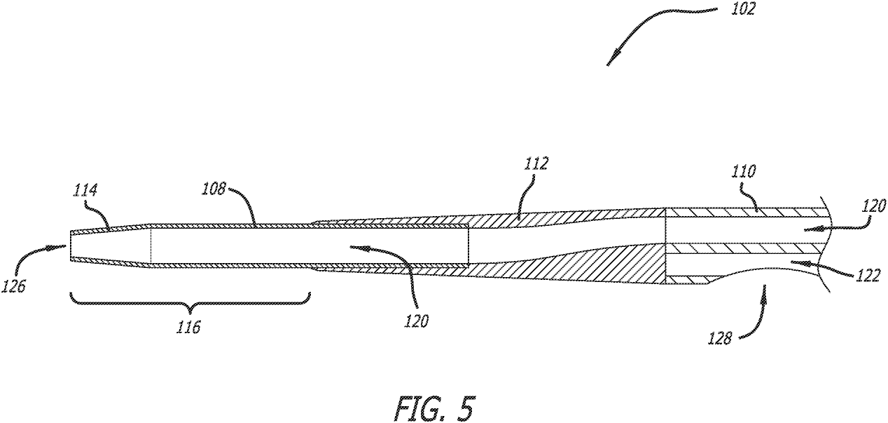

The first section 108 of the catheter tube 102 includes a distal tip 114 having a relatively short taper from an outer diameter of a distal end of the first section 108 to an outer diameter of a remainder of the first section 108. The taper of the distal tip 114 is configured for immediate dilation of tissue about a needle tract established with the introducer 132 up to the outer diameter of the remainder of the first section 108 of the catheter tube 102. As best shown in FIG. 5, the first section 108 of the catheter tube 102 also includes a proximal portion disposed in a bore of a distal portion of the junction 112 and fixedly coupled thereto such as by a solvent bond, an adhesive bond, or a heat weld.

The second section 110 of the catheter tube 102 includes a consistent outer diameter over its length from a distal end of the second section 110 to a proximal end of the second section 110. The consistent diameter of the second section 110 of the catheter tube 102 is configured for smooth insertion into the needle tract and targeted vasculature subsequent to any dilation by the first section 108 of the catheter tube 102 and the junction 112. The distal end of the second section 110 of the catheter has a flat face flush with a flat-faced proximal end of the junction 112 and fixedly coupled thereto such as by a solvent bond, an adhesive bond, or a heat weld.

The junction 112 includes a taper over its length from a distal end of the junction 112 to the proximal end of the junction 112. The taper of the junction 112 is configured for immediate dilation of the tissue about the needle tract from the outer diameter of a majority of the first section 108 of the catheter tube 102 to the outer diameter of the second section 110 of the catheter tube 102. An abluminal surface of the junction 112 smoothly transitions from an abluminal surface of the first section 108 of the catheter tube 102 to an abluminal surface of the second section 110 of the catheter tube 102 without edges that catch on skin when the catheter tube 102 is inserted into the needle tract. In addition to the edges being minimal to negligible, the edges can include solvent-interdiffused polymeric material of the polymeric materials from which the catheter tube 102 is formed, which smoothens the transitions from the first section 108 of the catheter tube 102 to the junction 112 and from the junction 112 to the second section 110 of the catheter tube 102. Notably, the junction 112 has a length approximately commensurate with a length of an exposed portion 116 of the first section 108 of the catheter tube 102 or between lengths of exposed portions of the first and second sections 108 and 110 of the catheter tube 102. As such, the length of the exposed portion 116 of the first section 108 of the catheter tube 102 is less than the length of the junction 112 up to approximately commensurate with the length of the junction 112.

The first section 108 of the catheter tube 102 is formed of a first polymeric material (e.g., a polytetrafluoroethylene, a polypropylene, or a polyurethane) having a first durometer. The second section 110 of the catheter tube 102 is formed of a second polymeric material (e.g., a polyvinyl chloride, a polyethylene, another polyurethane, or a silicone) having a second durometer less than the first durometer. For example, the first section 108 of the catheter tube 102 can be formed of a first polyurethane having the first durometer while the second section 110 of the catheter tube 102 can be formed of a second, different polyurethane (e.g., a same or different diisocyanate or triisocyanate reacted with a different diol or triol, a different diisocyanate or triisocyanate reacted with a same or different diol or triol, etc.) having the second durometer less than the first durometer. Indeed, polyurethanes are advantageous for the catheter tube 102 in that polyurethanes can be relatively rigid at room-temperature but become more flexible in vivo at body temperature, which reduces irritation to vessel walls and phlebitis. Polyurethanes are also advantageous in that they can be less thrombogenic than some other polymers. The junction 112 is formed of the second polymeric material or a third polymeric material (e.g., yet another polyurethane) having a third durometer less than the first durometer and greater than, approximately equal to, or less than the second durometer.

It should be understood the first durometer of the first polymeric material, the second durometer of the second polymeric material, and the third durometer of the third polymeric material can be on different scales (e.g., Type A or Type D). With this understanding, the second durometer of the second polymeric material or the third durometer of the third polymeric material might not be numerically less than the first durometer of the first polymeric material when the second durometer or the third durometer is less than the first durometer. Indeed, the hardness of the second polymeric material or the third polymeric material can still be less than the hardness of the first polymeric material as the different scales—each of which ranges from 0 to 100—are designed for characterizing different materials in groups of the materials having a like hardness.

In accordance with the first section 108 of the catheter tube 102, the second section 110 of the catheter tube 102, and the junction 112 between the first and second sections 108 and 110 of the catheter tube 102 set forth above, the catheter tube 102 possesses a column strength sufficient to prevent buckling of the catheter tube 102 when inserted into a needle tract established by a percutaneous puncture with the introducer 132 in accordance with the needle tract-establishing step set forth below. The column strength of the catheter tube 102 is also sufficient to prevent buckling of the catheter tube 102 when advanced through a vasculature of a patient without dilation of tissue about the needle tract or any blood vessels of the vasculature beforehand with a separate dilator.

The catheter tube 102 includes one or more catheter-tube lumens extending through the catheter tube 102; however, only one catheter-tube lumen typically extends from a proximal end of the catheter tube 102 to a distal end of the catheter tube 102 in a multiluminal RICC (e.g., a diluminal RICC, a triluminal RICC, a tetraluminal RICC, a pentaluminal RICC, a hexaluminal RICC, etc.). (See FIGS. 3-5.) Indeed, the first section 108 of the catheter tube 102 typically includes a single lumen therethrough as shown in FIG. 5.

The catheter hub 104 is coupled to a proximal portion of the catheter tube 102. The catheter hub 104 includes one or more catheter-hub lumens corresponding in number to the one-or-more catheter-tube lumens. The one-or-more catheter-hub lumens extends through an entirety of the catheter hub 104 from a proximal end of the catheter hub 104 to a distal end of the catheter hub 104.

Each extension leg of the one-or-more extension legs 106 is coupled to the catheter hub 104 by a distal portion thereof. The one-or-more extension legs 106 respectively include one or more extension-leg lumens, which, in turn, correspond in number to the one-or-more catheter-hub lumens. Each extension-leg lumen of the one-or-more extension-leg lumens extends through an entirety of the extension leg from a proximal end of the extension leg to a distal end of the extension leg.

Each extension leg of the one-or-more extension legs 106 includes a Luer connector 118 coupled to the extension leg, through which Luer connector 118 the extension leg and the extension-leg lumen thereof can be connected to another medical device and a lumen thereof.

As shown, the RICC 100 is a triluminal RICC including a set of three lumens; however, the RICC 100 is not limited to the set of the three lumens. The set of three lumens includes a primary lumen 120, a secondary lumen 122, and a tertiary lumen 124 formed of fluidly connected portions of three catheter-tube lumens, three catheter-hub lumens, and three extension-leg lumens. The primary lumen 120 has a primary-lumen aperture 126 in the distal end of the first section 108 of the catheter tube 102, which corresponds to the distal end of the catheter tube 102 and a distal end of the RICC 100. The secondary lumen 122 has a secondary-lumen aperture 128 in a side of the distal portion of the catheter tube 102. The tertiary lumen 124 has a tertiary-lumen aperture 130 in the side of the distal portion of the catheter tube 102 proximal of the secondary-lumen aperture 128.

### Introducers

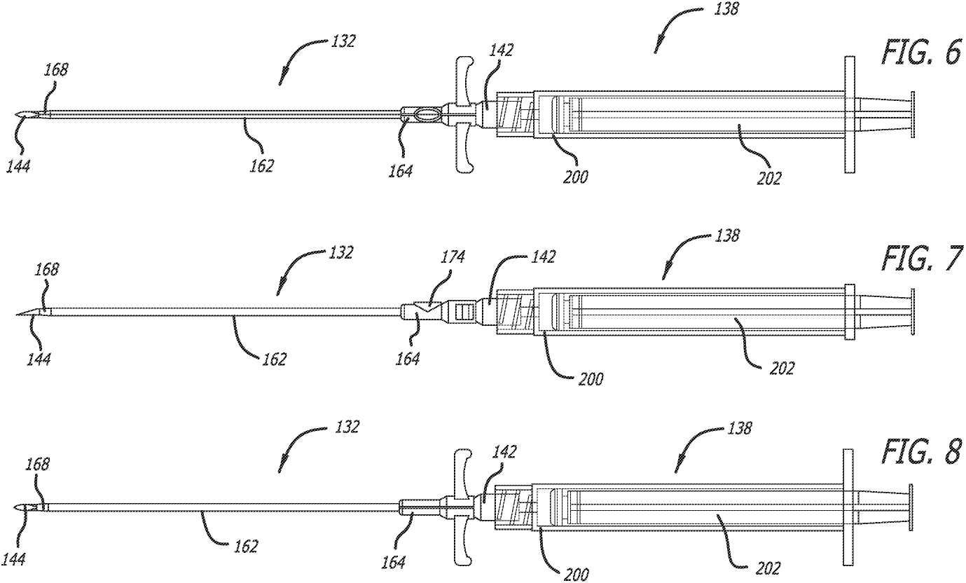

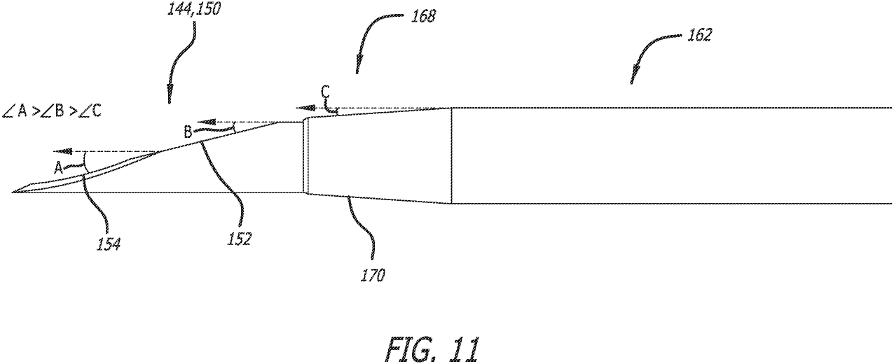

FIGS. 6-10 illustrate different views of an introducer 132 including an introducer needle 134 and a splittable introducer sheath 136 with and without a syringe 138 in accordance with some embodiments. FIG. 11 illustrates a distal portion of the introducer 132 in accordance with some embodiments.

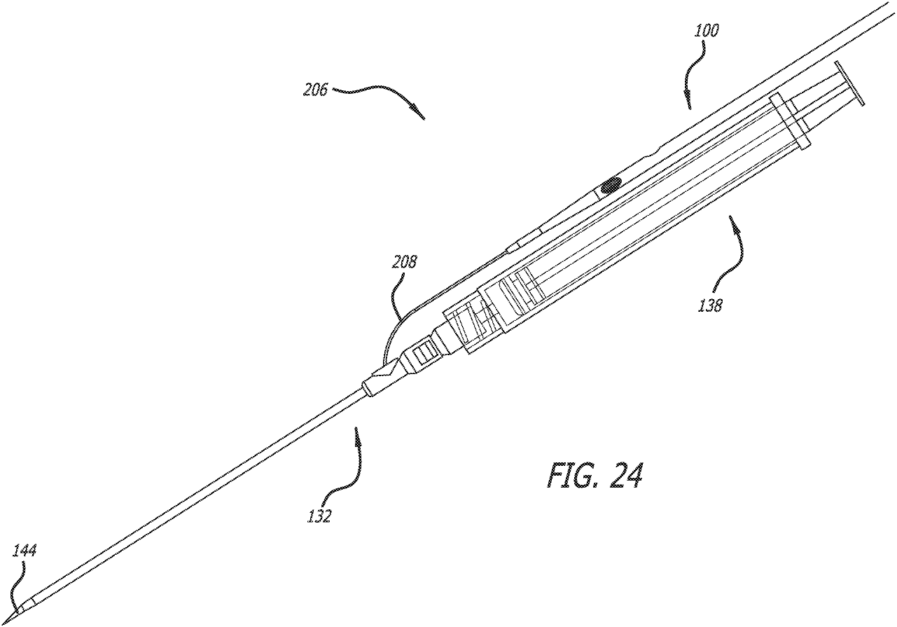

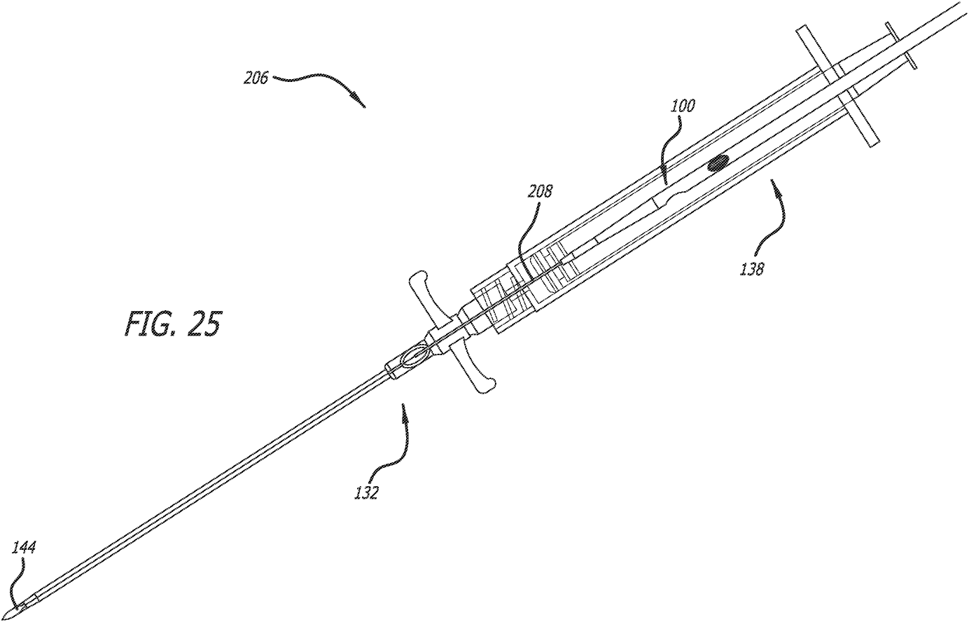

As shown, the introducer 132 includes the introducer needle 134 and the introducer sheath 136. In at least a ready-to-operate state of the introducer 132 configured for establishing a needle tract in accordance with the needle tract-establishing step set forth below, the introducer needle 134 is disposed in the introducer sheath 136. Said differently, the introducer sheath 136 is disposed over the introducer needle 134 in at least the ready-to-operate state of the introducer 132, which is shown in FIGS. 6-10 and 23-25.

FIG. 13 illustrates the introducer needle 134 of the introducer 132 in accordance with some embodiments.

The introducer needle 134 includes a needle shaft 140 and a needle hub 142 coupled to a proximal portion of the needle shaft 140, particularly a proximal-end portion of the needle shaft 140.

The needle shaft 140 includes a needle tip 144 in a distal portion of the needle shaft 140 and a longitudinal needle slot 146.

The needle tip 144 includes a bevel 150 having a primary bevel 152 and secondary a secondary or tip bevel 154 distal of the primary bevel 152. A tip-bevel angle (see ∠A in FIG. 11) of the tip bevel 154 is greater than a primary-bevel angle (see ∠B in FIG. 11) of the primary bevel 152 such that the bevel 150 provides a smooth transition over the needle tip 144. Such a needle tip is thusly configured for establishing a needle tract from an area of skin into a blood-vessel lumen of a patient in accordance with the needle tract-establishing step of the method set forth below.

The needle slot 146 distally extends from a proximal portion of the needle shaft 140 through the needle tip 144, thereby forming a needle channel along a majority of a length of the introducer needle 134 as opposed to a needle lumen therethrough. (Notably, the introducer 132 includes an introducer lumen; however, the introducer 132 lumen results from the introducer sheath 136 over the needle shaft 140, wherein the introducer sheath 136 seals the needle slot 146 thereunder forming the introducer 132 lumen from the needle channel.) The needle slot 146 has a width sized in accordance with the outer diameter of the access guidewire 208, which outer diameter can vary as set forth below to prevent proximal withdrawal of the access guidewire 208 from the needle shaft 140 while still allowing the access guidewire 208 to pass through the needle shaft 140 during the introducer needle-withdrawing step of the method set forth below. Indeed, various configurations of the needle slot 146 and the access guidewire 208 that prevent proximal withdrawal of the access guidewire 208 from the needle shaft 140 are set forth below with respect to the introducer assembly 220.

The needle hub 142 can include a non-tapered but optionally chamfered male needle-hub connector 156 in a distal portion of the needle hub 142 as well as a tapered female needle-hub connector 158 in a proximal portion of the needle hub 142. The male needle-hub connector 156 is configured to form a fluid-tight connection with the female sheath-hub connector 182 set forth below in at least the ready-to-operate state of the introducer 132. Indeed, the male needle-hub connector 156 can include an ‘O′-ring 160 disposed in a circumferential groove around the male needle-hub connector 156 configured to form the fluid-tight connection. The female needle-hub connector 158 is configured to form a fluid-tight connection with the syringe tip 204 of the syringe 138 set forth below.

FIG. 14 illustrates the introducer sheath 136 of the introducer 132 in accordance with some embodiments.

The introducer sheath 136 includes a splittable sheath body 162 and a splittable sheath hub 164 around a proximal portion of the sheath body 162 and coupled thereto. The introducer sheath 136 is configured such that an entirety of the introducer sheath 136 is disposed over the introducer needle 134 in at least the ready-to-operate state of the introducer 132. Indeed, an entirety of the sheath body 162 is disposed over the needle shaft 140 and the sheath hub 164 is coupled to the proximal portion of the sheath body 162 such that the male needle-hub connector 156 of the needle hub 142 forms a fluid-tight connection with the female sheath-hub connector 182 of the sheath hub 164 in the ready-to-operate state of the introducer 132.

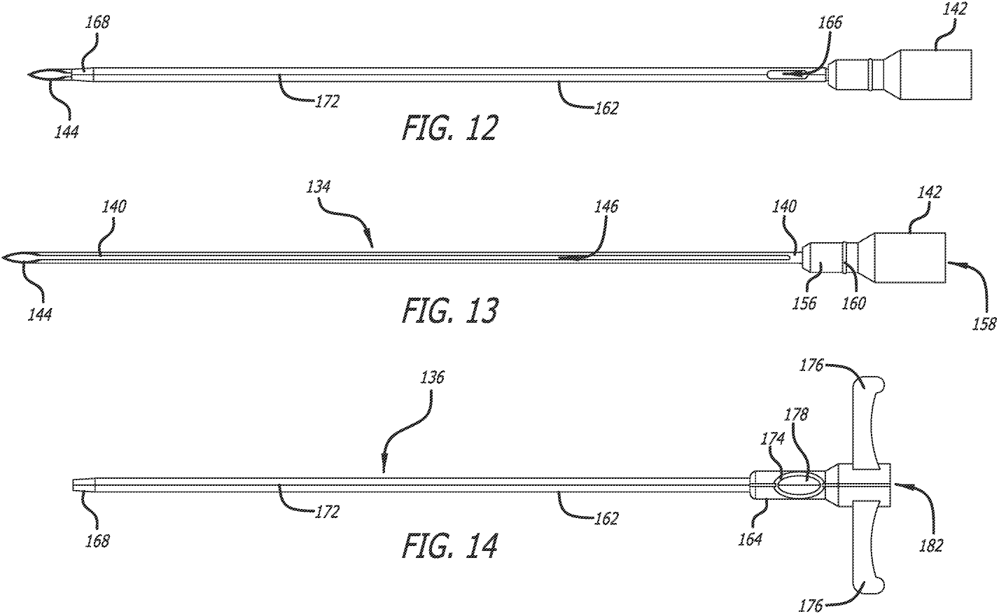

The sheath body 162 includes a sheath-body opening 166 in the proximal portion of the sheath body 162 and a sheath tip 168 in a distal portion of the sheath body 162. (See FIG. 12 for the sheath-body opening 166.) Notwithstanding the sheath-body opening 166, which is covered by the sheath hub 164 coupled to the proximal portion of the sheath body 162, the sheath body 162 is configured to seal the needle shaft 140 thereunder such that a vacuum can be drawn through the introducer 132 in at least the ready-to-operate state of the introducer 132 shown in FIGS. 6-10 and 23-25.

The sheath-body opening 166 has a width approximately commensurate with a width of the needle slot 146 in the needle shaft 140, which, as set forth above, can vary in accordance with the outer diameter of the access guidewire 208 to prevent proximal withdrawal of the access guidewire 208 from the needle shaft 140. The sheath-body opening 166 is thusly configured to allow the access guidewire 208 to pass therethrough and into the needle slot 146 of the needle shaft 140.

The sheath tip 168 includes a taper 170 from an outer diameter of the needle shaft 140 up to an outer diameter of a majority of the sheath body 162. Said differently, the taper 170 is from the outer diameter of the majority of the sheath body 162 down to the outer diameter of the needle shaft 140. The taper 170 has a taper angle (see ∠C in FIG. 11) less than the primary-bevel angle (see ∠B in FIG. 11) of the primary bevel 152 of the needle tip 144, which, in turn, is less than the tip-bevel angle (see ∠A in FIG. 11) of the tip bevel 154 of the needle tip 144. The sheath tip 168 including such a taper is configured to provide a smooth transition from the needle tip 144 to the sheath body 162 in the ready-to-operate state of the introducer 132.

The sheath body 162 can also include one or more longitudinal body faults 172 (e.g., grooves, lines of weakened material, etc.) such as a pair of the body faults 172 extending along opposite sides of the sheath body 162 if the sheath body 162 is not formed of a material configured to split or propagate a split. For example, the sheath body 162 can be formed of a polymeric material such as polytetrafluoroethylene, which facilitates smooth, consistent splitting of the sheath body 162 without the body faults 172 along the sides of the sheath body 162. When present, the one-or-more body faults 172 include at least a primary body fault extending along at least a portion of a primary side of the introducer sheath 136 including the port 174. Indeed, the primary body fault can extend along an entirety of the sheath body 162 as shown in FIG. 12. When the pair of the body faults 172 is present, a secondary body fault extends along at least a portion of a secondary side of the introducer sheath 136 opposite the primary side. While not shown, the secondary body fault can also extend along the entirety of the sheath body 162.

The sheath hub 164 includes a splittable valved port 174 in the primary side of the introducer sheath 136 and a pair of tabs 176 extending from the sheath hub 164 proximal of the port 174.

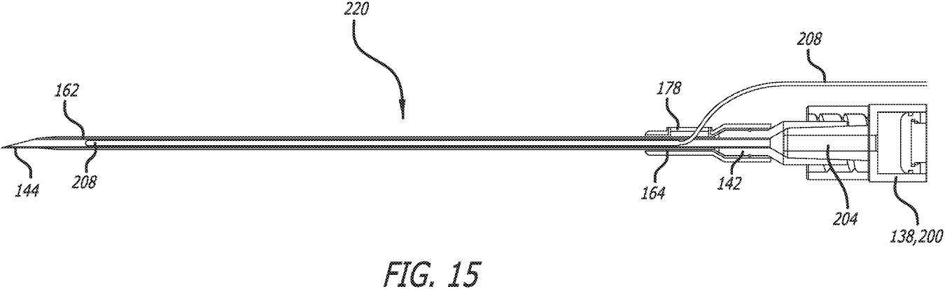

The port 174 lies over the sheath-body opening 166 in the proximal portion of the sheath body 162. While the port 174 has a length approximately commensurate with a length of the sheath-body opening 166, a width of the port 174 is wider than the width of the sheath-body opening 166 to accommodate a valve 178 such as a partially or fully split septum compressed in the port 174 over the sheath-body opening 166 of the sheath body 162. As best shown in FIGS. 15, 24, and 25, the valve 178 is configured to allow the access guidewire 208 to pass through the valve 178, through the port 174, and into the sheath-body opening 166 of the sheath body 162, notably, while maintaining fluid-tight access to a blood-vessel lumen of a patient with the introducer 132 and, thereby, enabling the blood-aspirating step of the method set forth below. Such a valve is also configured to split with a remainder of the sheath hub 164 when splitting the introducer sheath 136 away from the access guidewire 208 in accordance with the introducer sheath-splitting step of the method set forth below.

The tabs 176 radially extend from opposite sides of the sheath hub 164 orthogonal to the primary and secondary sides of the introducer sheath 136, which, in turn, positions the tabs 176 orthogonal to at least the primary body fault when present in the primary side of the introducer sheath 136. Such tabs are configured to split the sheath hub 164 when the tabs 176 are pulled apart from each other in accordance with the introducer sheath-splitting step of the method set forth below. Notwithstanding the foregoing, the tabs 176 need not be 180° apart from each other as shown. Indeed, the tabs 176 can radially extend from sides of the sheath hub 164 that are less than 180° apart, including less than 135° apart, such as less than 90° apart, for example, about 67.5° apart, with the port 174 opposite a vertex of an internal angle formed between the tabs 176. Such tabs are configured to split the sheath hub 164 when the tabs 176 are pushed into each other such as by pinching the tabs 176 together with a single hand.

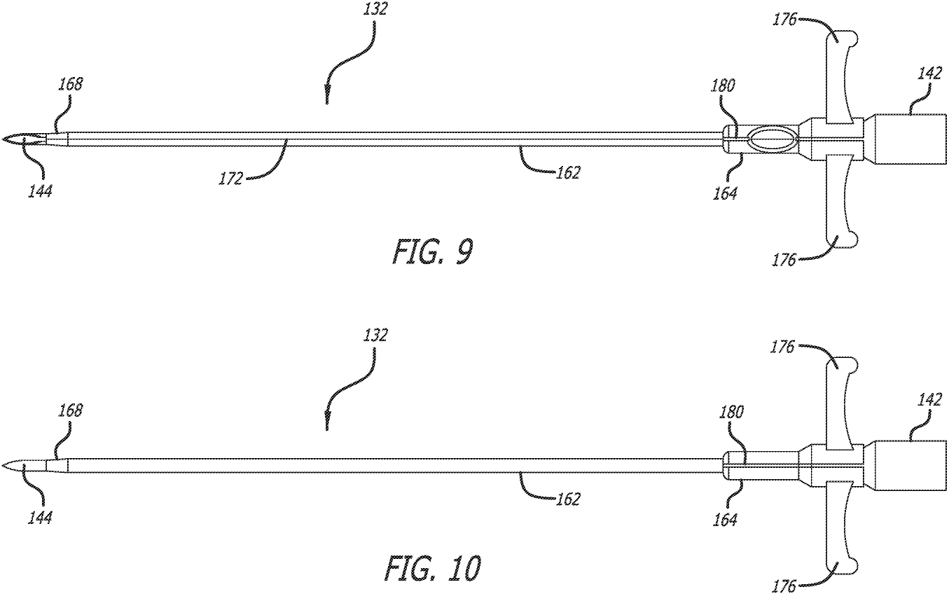

The sheath hub 164 can also include a pair of longitudinal hub faults 180 (e.g., grooves, lines of weakened material, etc.) extending along opposing sides of the sheath hub 164. When present, the hub faults 180 include a primary hub fault and a secondary hub fault. The primary hub fault extends along at least a portion of the primary side of the introducer sheath 136 (i.e., the side of the introducer sheath 136 including the port 174). Indeed, the primary hub fault can extend along an entirety of the sheath hub 164 as shown in FIG. 9. Likewise, the secondary hub fault extends along at least a portion of the secondary side of the introducer sheath 136 opposite the primary side. As shown in FIG. 10, the secondary hub fault can also extend along the entirety of the sheath hub 164. The sheath hub 164 is configured to split along both the primary and secondary hub faults 180 when the tabs 176 are pulled apart (or pinched together) for propagating splits resulting from splitting the sheath hub 164 along the sheath body 162 in the primary and secondary sides of the introducer sheath 136.

The sheath hub 164 can also include a non-tapered female sheath-hub connector 182 in a proximal portion of the sheath hub 164. The female sheath-hub connector 182 is configured to form a fluid-tight connection with the male needle-hub connector 156 set forth above in at least the ready-to-operate state of the introducer 132. Such non-tapered connectors are less likely to get stuck together than tapered connectors (e.g., Luer-tapered connectors), which is advantageous for the introducer needle-withdrawing step of the method set forth below, as the introducer needle-withdrawing step includes withdrawing the introducer needle 134 from the introducer 132 and leaving the introducer sheath 136 in place in a blood-vessel lumen of a patient.

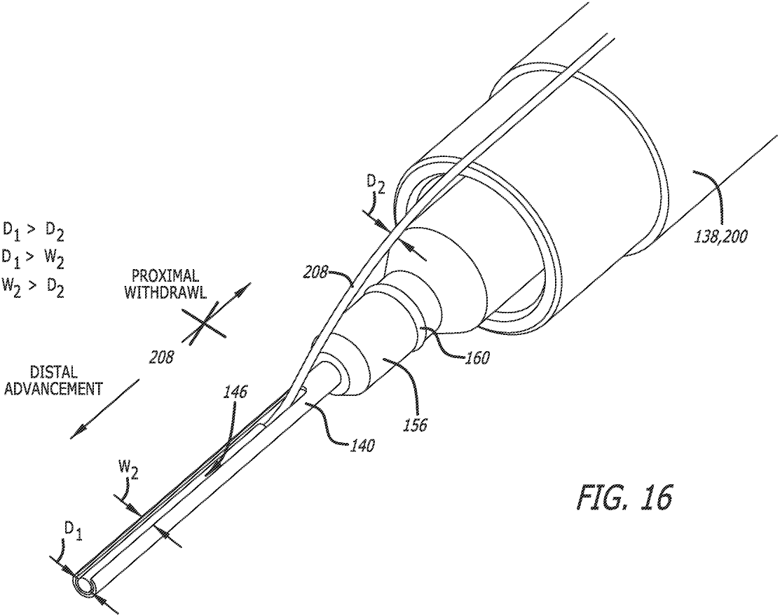

FIGS. 15 and 20-22 illustrate different views of a distal portion of an introducer assembly 220 in accordance with some embodiments. FIGS. 16-19 illustrate different views of the distal portion of the introducer assembly 220 in accordance with the same or different embodiments, wherein the introducer sheath 136 is removed from the introducer needle 134 for expository expediency.

As shown, the introducer 132 can form the introducer assembly 220 with an access guidewire 208, the syringe 138, or both the access guidewire 208 and the syringe 138. Notably, the access guidewire-advancing step of the method set forth below is not required for securing blood-vessel access in view of other techniques such as advancing the introducer sheath 136 into a blood-vessel lumen of a patient to secure the blood-vessel access. As such, the access guidewire 208 need not be included in the introducer assembly 220 in a ready-to-operate state of the introducer assembly 220 or otherwise. In addition, the blood-aspirating step of the method set forth below is not required for confirming a needle tract extends into the blood-vessel lumen of the patient in view of other techniques such as ensuring blood flashes back into the introducer 132. As such, the syringe 138 need not be included in the introducer assembly 220 in the ready-to-operate state of the introducer assembly 220 or otherwise. Also, it should be understood the introducer assembly 220 can be used separately from the RICC 100 to introduce CVCs into patients. Indeed, the needle-tract establishing step of the method set forth below can be performed with the introducer assembly 220, and, after securing blood-vessel access with the access guidewire 208 or the introducer sheath 136, tissue about the needle tract can be dilated with a dilator for subsequent CVC introduction.

The access guidewire 208 includes a proximal portion including a proximal end, which proximal end can include the stop 210 set forth below to prevent over advancement of the access guidewire 208. The access guidewire 208 also includes a distal portion including a distal-end portion, which distal-end portion, in turn, includes, a distal end of the access guidewire 208. As shown in FIG. 15 and set forth in detail below, the proximal portion of the access guidewire 208 is disposed in the RICC 100 in the ready-to-operate state of the RICC insertion assembly 206. The distal-end portion of the access guidewire 208 is disposed in the introducer 132 in both the ready-to-operate state of the introducer assembly 220 and the ready-to-operate state of the RICC insertion assembly 206 when the access guidewire 208 is present in the foregoing assemblies.

The access guidewire 208 also includes an outer diameter (e.g., D₁ or D₂ as shown in FIGS. 16-18) that can vary over at least the distal portion of the access guidewire 208. As set forth above, the needle slot 146 has a width (e.g., W₁ or W₂ as shown in FIGS. 16-18) sized in accordance with the outer diameter of the access guidewire 208 to prevent proximal withdrawal of the access guidewire 208 from the needle shaft 140 while still allowing the access guidewire 208 to pass through the needle shaft 140 during the introducer needle-withdrawing step of the method set forth below. Indeed, various configurations of the access guidewire 208 and the needle slot 146 that prevent proximal withdrawal of the access guidewire 208 from the needle shaft 140 are set forth immediately below.

The outer diameter (e.g., D₁ as shown in FIGS. 16-18) of the distal-end portion of the access guidewire 208 can be greater than the width (e.g., W₂ as shown in FIGS. 16-18) of at least a proximal-end portion of the needle slot 146 such that at least the proximal-end portion of the needle slot 146 catches the distal-end portion of the access guidewire 208 when the access guidewire 208 is proximally withdrawn, thereby preventing proximal withdrawal of the access guidewire 208 from the needle shaft 140 and, thus, the introducer needle 134. (See FIGS. 16-19.) The needle slot 146 including the proximal-end portion thereof can, in turn, have a constant width (e.g., W₂ as shown in FIG. 16) less than the outer diameter (e.g., D₁ as shown in FIG. 16) of the distal-end portion of the access guidewire 208. In other words, the proximal-end portion of the needle slot 146 can have a same width as a remainder of the needle slot 146, which width is less than the outer diameter of the of the distal-end portion of the access guidewire 208. (See FIG. 16.) With such a configuration, distal advancement of the access guidewire 208 is limited up to the stop 210 of the access guidewire 208; proximal withdrawal of the access guidewire 208 is prevented by both the distal-end portion of the access guidewire 208 and the proximal-end portion of the needle slot 146.

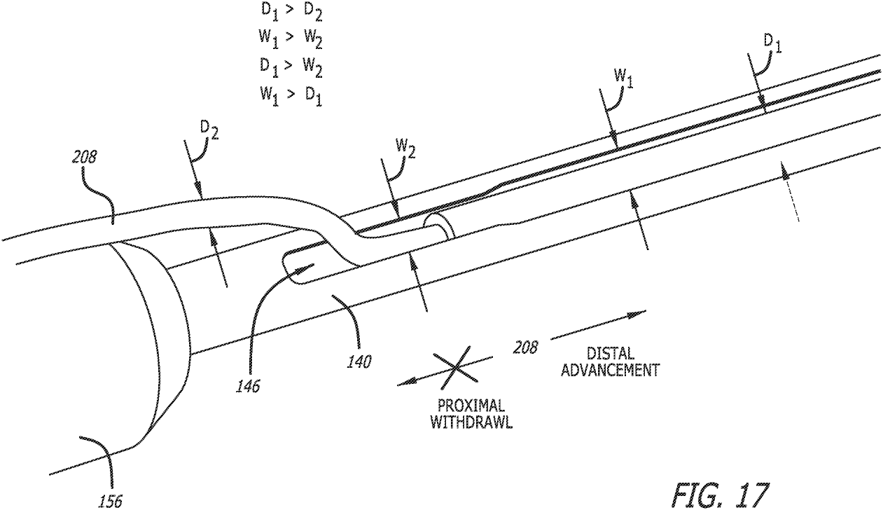

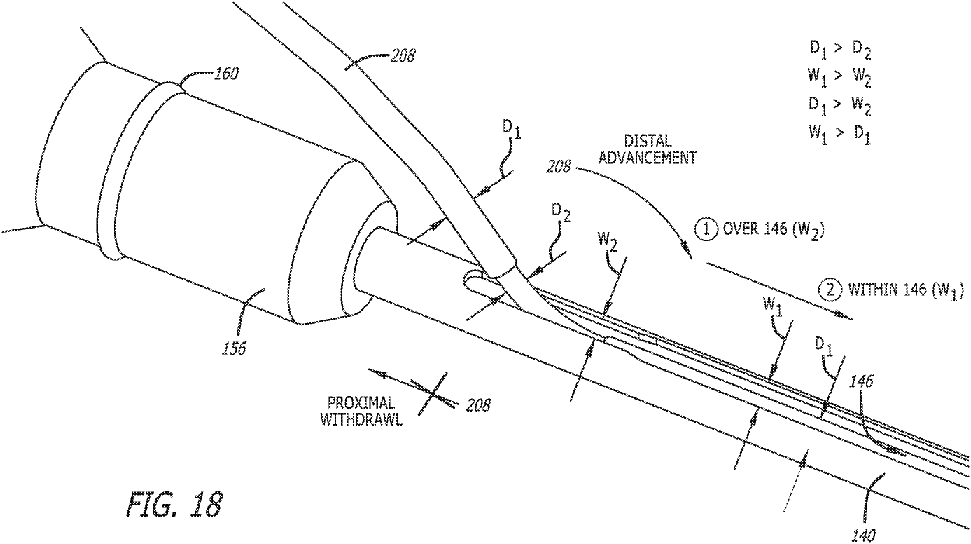

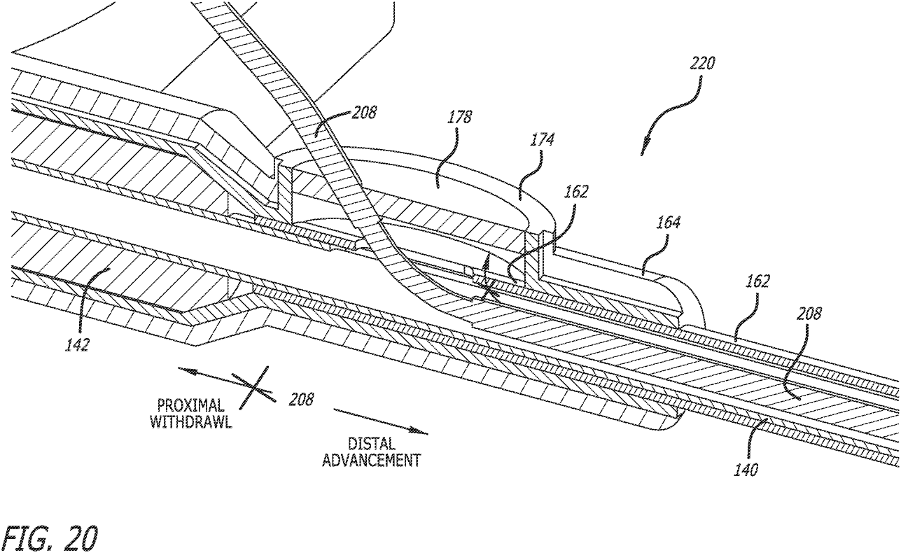

Following on the foregoing in which the outer diameter (e.g., D₁ as shown in FIGS. 16-18) of the distal-end portion of the access guidewire 208 can be greater than the width (e.g., W₂ as shown in FIGS. 16-18) of at least the proximal-end portion of the needle slot 146, the proximal-end portion of the needle slot 146 can alternatively be narrower than the remainder of the needle slot 146. In other words, the remainder of the needle slot 146 can have a greater width (e.g., W₁ as shown in FIGS. 17 and 18) than the proximal-end portion of the needle slot 146, which facilitates loading the access guidewire 208—particularly the distal-end portion thereof-into the needle shaft 140 when assembling the introducer assembly 220. (See FIGS. 17-19.) The sheath-body opening 166 of the introducer sheath 136, the port 174 including the valve 178 thereof, or the sheath hub 164 (e.g., housing) therearound can be configured to direct (e.g., by interference) the distal-end portion of the access guidewire 208 into the proximal-end portion of the needle slot 146 when the access guidewire 208 is proximally withdrawn, thereby preventing proximal withdrawal of the access guidewire 208 from the needle shaft 140. Indeed, as shown in FIG. 20 the sheath-body opening 166 extends over the proximal-end portion of the needle slot 146 without substantially extending over the remainder of the needle slot 146 such that the sheath body 162 prevents the proximal withdrawal of the access guidewire 208 from the needle shaft 140 by interfering with the distal-end portion of the access guidewire 208. Also, as shown in FIG. 22, the port 174 is over the remainder of the needle slot 146 exposed by the sheath-body opening 166, which port 174 exclusively directs the access guidewire 208 into the proximal-end portion of the needle slot 146, thereby preventing the proximal withdrawal of the access guidewire 208 from the needle shaft 140. Alternatively or additionally, the sheath hub 164 extends over the remainder of the needle slot 146 exposed by the sheath-body opening 166 such that the sheath hub 164 prevents the proximal withdrawal of the access guidewire 208 from the needle shaft 140 by interfering with the distal-end portion of the access guidewire 208. Like that set forth above, distal advancement of the access guidewire 208 is limited up to the stop 210 of the access guidewire 208 with such a configuration; proximal withdrawal of the access guidewire 208 is prevented by both the distal-end portion of the access guidewire 208 and the proximal-end portion of the needle slot 146.

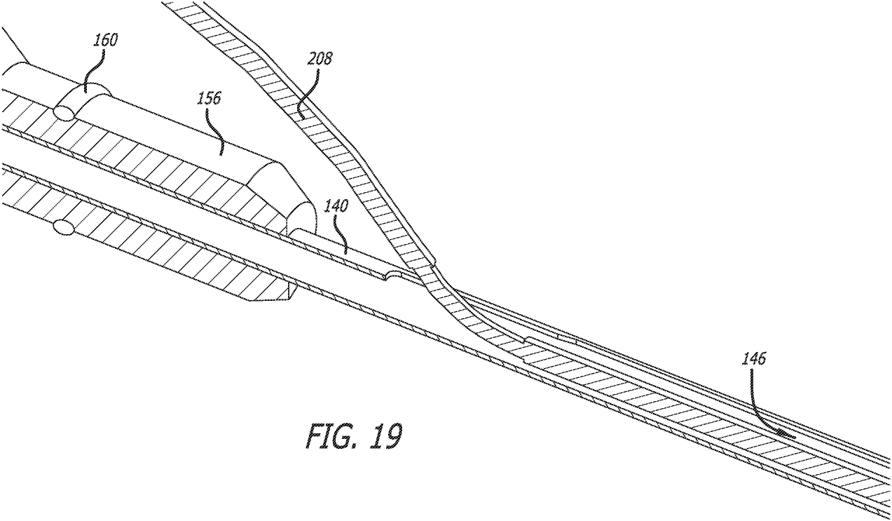

The outer diameter (e.g., D₂ as shown in FIGS. 16 and 17) of the proximal portion of the access guidewire 208 can be less than the outer diameter (e.g., D₁ as shown in FIGS. 16-18) of the distal-end portion of the access guidewire 208, thereby allowing the proximal portion of the access guidewire 208 to be easily advanced into the foregoing constant-width needle slot 146 or the narrower proximal-end portion of the needle slot 146 and, subsequently, a blood-vessel lumen in the access guidewire-advancing step of the method set forth below. (See FIGS. 15-17.) However, the outer diameter (e.g., D₁ as shown in FIG. 18) of the proximal portion of the access guidewire 208 can alternatively be commensurate with the outer diameter (e.g., D₁ as shown in FIG. 18) of the distal-end portion of the access guidewire 208. (See FIGS. 18-22.) So configured, the outer diameter (e.g., D₁ as shown in FIG. 18) of an intermediary portion of the access guidewire 208 between the proximal portion of the access guidewire 208 and the distal-end portion of the access guidewire 208 is less than that of the proximal portion of the access guidewire 208 and the distal-end portion of the access guidewire 208. In certain embodiments such as those of FIGS. 18 and 19, the proximal portion of the access guidewire 208, namely that proximal of the intermediary portion of the access guidewire 208, slides over the narrower proximal-end portion of the needle slot 146 and into the remainder of the needle slot 146 during the access guidewire-advancing step of the method set forth below (optionally, facilitated by a taper from the proximal portion of the access guidewire 208 into the intermediate portion of the access guidewire 208); however, when the access guidewire 208 is proximally withdrawn, the intermediary portion of the access guidewire 208 slides into and catches the narrower proximal-end portion of the needle slot 146, thereby preventing proximal withdrawal of the access guidewire 208 from the needle shaft 140. Like that set forth above, distal advancement of the access guidewire 208 is limited up to the stop 210 of the access guidewire 208 with such a configuration; proximal withdrawal of the access guidewire 208 is prevented by both the distal-end portion of the access guidewire 208 and the proximal-end portion of the needle slot 146.

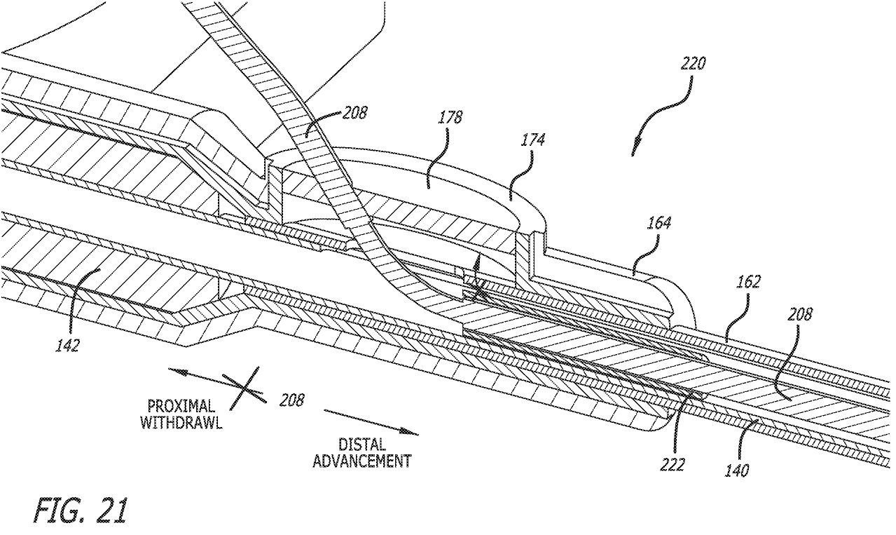

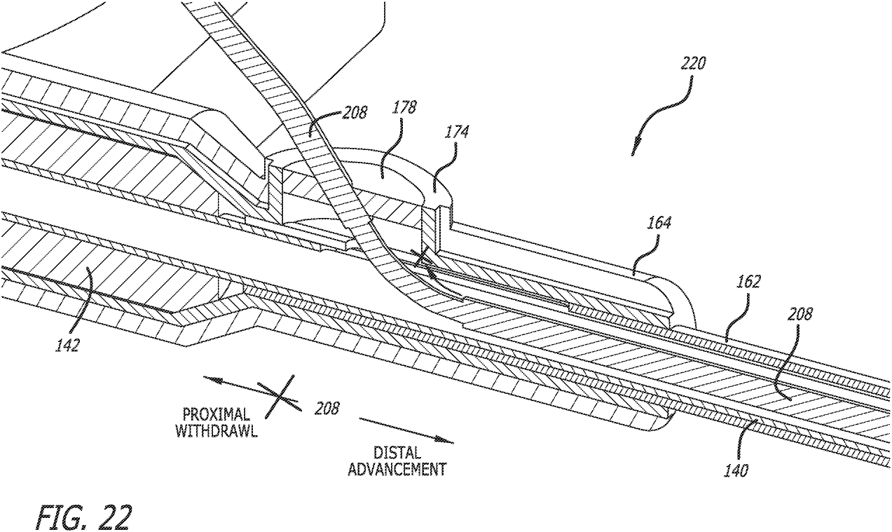

Notwithstanding the foregoing, a proximal portion of the distal-end portion of the access guidewire 208 can include a rigid tubular fitting 222 thereover, thereby preventing the proximal portion of the distal-end portion of the access guidewire 208 from bending. (See FIG. 21.) By preventing the proximal portion of the distal-end portion of the access guidewire 208 from bending, the distal-end portion of the access guidewire 208 can be directed to engage the proximal-end portion of the needle slot 146 instead of the remainder of the needle slot 146.

The access guidewire 208 can include a guidewire tip in the distal-end portion of the access guidewire 208, which adopts a ‘J’ shape configured to prevent puncturing a back wall of a blood vessel. Such a guidewire tip assumes a straightened state in the ready-to-operate state of the RICC insertion assembly 206 and a curved state when the guidewire tip is advanced beyond the needle tip 144 (e.g., advanced into a blood-vessel lumen) in one-or-more operating states of the RICC insertion assembly 206 in which the access guidewire 208 is deployed.

The access guidewire 208 can further include a bare-wire portion and a wound-wire portion distal of the bare-wire portion, proximal of the bare-wire portion, or both. While not shown, the bare-wire portion, when present, distally extends through the valve 178 of the port 174 of the sheath hub 164 in at least the ready-to-operate state of the RICC insertion assembly 206 such that the valve 178 forms a fluid-tight seal around the bare-wire portion of the access guidewire 208. Notably, the foregoing bare-wire portion can instead be a flat-wound or ground-wound portion of the access guidewire 208, wherein the flat-wound portion includes windings of a tape instead of a round wire, and wherein the ground-wound portion includes windings of a round wire ground down to flatten the windings.

The syringe 138 includes a barrel 200, a plunger 202 disposed in the barrel 200, and a tapered male syringe tip 204 extending from a distal portion of the syringe 138. The syringe tip 204 is configured to insert into the female needle-hub connector 158 set forth above. Such a syringe is useful for at least aspirating blood in accordance with the blood-aspirating step of the method set forth below.

### RICC Insertion Assemblies

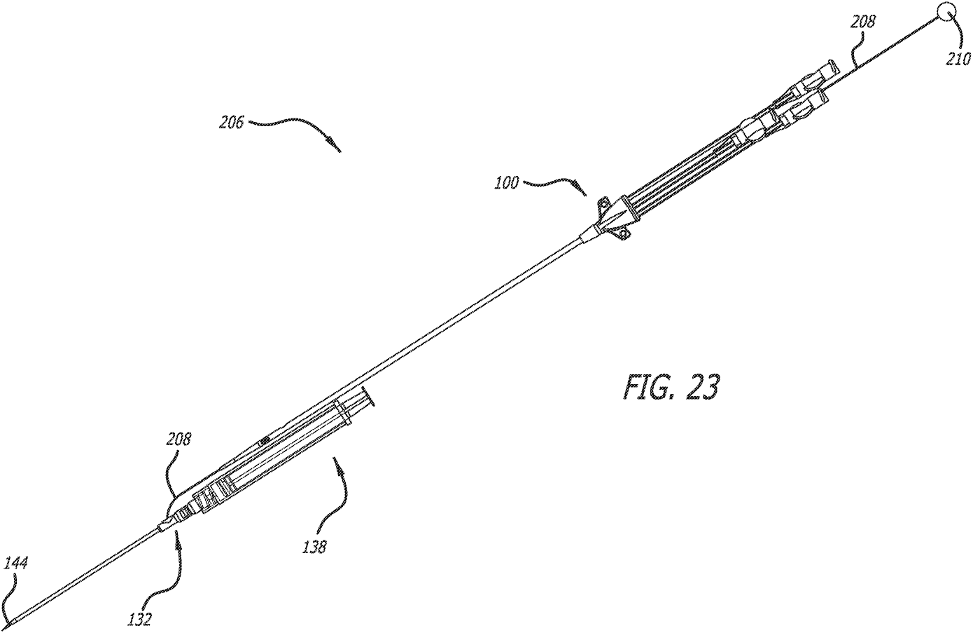

FIGS. 23-25 illustrate different views of a RICC insertion assembly 206 in accordance with some embodiments.

As shown, the RICC insertion assembly 206 includes the RICC 100, the introducer 132, and the access guidewire 208 disposed in both the RICC 100 and the introducer 132 in at least a ready-to-operate state of the RICC insertion assembly 206. Specifically, the proximal portion of the access guidewire 208 is disposed in the RICC 100 and the distal-end portion of the access guidewire 208 is disposed in the introducer 132 in the ready-to-operate state of the RICC insertion assembly 206.

Description for the RICC 100 and the introducer 132 is set forth above. Notably, in at least the ready-to-operate state of the RICC insertion assembly 206, the introducer 132 is also in its ready-to-operate state, description for which is also set forth above.

The access guidewire 208 extends along an entirety of the primary lumen 120 of the RICC 100, through the valve 178 (e.g., the septum) of the port 174 of the sheath hub 164 of the introducer sheath 136, along the introducer lumen of the introducer 132, and to a location in the introducer 132 proximal of the needle tip 144. The location of the access guidewire 208 proximal of the needle tip 144 is advantageous for immediate advancement of a distal end of the access guidewire 208 through a remainder of the introducer 132 and into a blood-vessel lumen in accordance with the access guidewire-advancing step of the method set forth below. Notably, the access guidewire 208 includes a stop 210 (e.g., a guidewire hub) about the proximal end of the access guidewire 208 configured to prevent over advancement of the access guidewire 208 during the access guidewire-advancing step and, thereby, loss of the access guidewire 208 within the RICC insertion assembly 206 or a patient, which can cause a guidewire embolism.

As set forth above for the introducer 132, the RICC insertion assembly 206 can further include the syringe 138 as shown. Again, such a syringe is useful for at least aspirating blood in accordance with the blood-aspirating step of the method set forth below.

### RICC Insertion Kits

While not shown, a RICC insertion kit includes the RICC 100, the introducer 132, and the access guidewire 208, instructions for use of the RICC insertion kit, and packaging. The foregoing components of the RICC insertion assembly 206 are optionally assembled into a substantially ready-to-operate state of the RICC insertion assembly 206 in the RICC insertion kit and disposed in the packaging thereof. The instructions for use of the RICC insertion kit are either disposed in the packaging or printed on the packaging.

### Methods

Methods include at least a method for inserting the RICC 100 into a blood-vessel lumen of a patient. Such a method includes one or more steps selected from an insertion assembly-acquiring step, a needle tract-establishing step, an access guidewire-advancing step, a blood-aspirating step, an introducer needle-withdrawing step, an introducer sheath-splitting step, a RICC-advancing step, an access guidewire-withdrawing step, a maneuver guidewire-advancing step, an additional RICC-advancing step, and a maneuver guidewire-withdrawing step.

The insertion assembly-acquiring step includes acquiring the RICC insertion assembly 206 including the RICC 100, the introducer 132, and the access guidewire 208 disposed in both the RICC 100 and the introducer 132. Optionally, the RICC insertion assembly 206 is acquired in a substantially ready-to-operate state of the RICC insertion assembly 206. If the RICC insertion assembly 206 is not acquired in the substantially ready-to-operate state thereof, the RICC insertion assembly 206 can be adjusted as needed to put the RICC insertion assembly 206 in the ready-to-operate state thereof before performing the needle tract-establishing step.

The needle tract-establishing step includes establishing a needle tract from an area of skin to the blood-vessel lumen with the introducer 132. The needle tract-establishing step can include drawing a slight vacuum with the syringe 138 while establishing the needle tract such that blood flashes back into at least the introducer needle 134 upon establishing the needle tract. In accordance with drawing such a vacuum, the needle tract-establishing step can include ensuring blood flashes back along the needle slot 146 of the introducer needle 134 if the sheath body 162 is formed of a clear polymeric material, into the needle hub 142 of the introducer needle 134 if the needle hub 142 is formed of a clear polymeric material, into the syringe tip 204, into the barrel 200 of the syringe 138, or a combination thereof, thereby confirming the needle tract extends into the blood-vessel lumen.

The access guidewire-advancing step includes advancing the distal end of the access guidewire 208 from its initial location in the introducer 132 proximal of the needle tip 144 into the blood-vessel lumen to secure blood-vessel access for the RICC-advancing step. The remainder of the access guidewire 208 proximally extends along the introducer lumen of the introducer 132, through the valve 178 of the port 174 in the side of the sheath hub 164, along the entirety of the primary lumen 120 of the RICC 100, and out a proximal end of the RICC 100. Again, the access guidewire 208 includes the stop 210 (e.g., the guidewire hub) about the proximal end of the access guidewire 208 configured to prevent over advancement of the access guidewire 208 during the access guidewire-advancing step.

The blood-aspirating step includes aspirating blood with the syringe 138 coupled to the introducer needle 134 for confirmation the needle tract extends into the blood-vessel lumen before withdrawing the introducer needle 134 from the introducer sheath 136 in the introducer needle-withdrawing step. Notably, the needle shaft 140 has an inner diameter sized in accordance with the outer diameter of the distal-end portion of the access guidewire 208 to allow blood flashback and aspiration. This advantageously enables the blood-aspirating step to be performed after the access guidewire-advancing step while the access guidewire 208 secures or maintains the blood-vessel access for the RICC-advancing step.

The introducer needle-withdrawing step includes withdrawing the introducer needle 134 from the introducer sheath 136 leaving both the introducer sheath 136 and the access guidewire 208 in place in the blood-vessel lumen. Again, the introducer needle 134 includes the needle slot 146 extending from the proximal portion of the needle shaft 140 through the needle tip 144, thereby allowing removal of the introducer needle 134 from the introducer sheath 136 while the access guidewire 208 remains in place in the blood-vessel lumen.

The introducer sheath-splitting step includes splitting the introducer sheath 136 away from the access guidewire 208 to remove split portions of the introducer sheath 136 from the blood-vessel lumen. Such splitting includes splitting the sheath hub 164 and the port 174 thereof by pulling apart the tabs 176 extending from the sheath hub 164. The introducer sheath-splitting step also includes propagating splits resulting from the splitting of the sheath hub 164 along the sheath body 162 of the introducer sheath 136. In this way, the introducer sheath 136 is split away from the access guidewire 208 to remove the split portions of the introducer sheath 136 from the blood-vessel lumen.

The RICC-advancing step includes advancing the distal portion of the catheter tube 102 of the RICC 100 over the access guidewire 208 and into the blood-vessel lumen, thereby inserting the RICC 100 into the blood-vessel lumen.

The access guidewire-withdrawing step includes withdrawing the access guidewire 208 leaving the catheter tube 102 in place in the blood-vessel lumen. The access guidewire-withdrawing step also includes removing the access guidewire 208 from the primary lumen 120 of the RICC 100 so the maneuver guidewire-advancing step can be performed.

The maneuver guidewire-advancing step includes advancing a maneuver guidewire into the blood-vessel lumen by way of the primary lumen 120 of the RICC 100. In accordance with the maneuver guidewire-advancing step, a distal portion of the maneuver guidewire can be advanced to a lower ⅓ of an SVC of a heart of the patient.

The additional RICC-advancing step includes advancing the distal portion of the catheter tube 102 farther into the blood-vessel lumen over the maneuver guidewire to the lower ⅓ of the SVC of the patient.

The maneuver guidewire-withdrawing step includes withdrawing the maneuver guidewire leaving the catheter tube 102 in place in the lower ⅓ of the SVC.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. An introducer assembly, comprising:
an introducer including:
an introducer needle including a needle shaft having a longitudinal needle slot extending from a proximal portion of the needle shaft through a distal needle tip; and
a splittable introducer sheath including a sheath body over the needle shaft sealing the needle slot thereunder but for a sheath-body opening in a proximal portion of the sheath body; and
an access guidewire including a distal-end portion disposed in the introducer, the distal-end portion of the access guidewire having an outer diameter greater than at least a proximal-end portion of the needle slot such that the proximal-end portion of the needle slot catches the distal-end portion of the access guidewire preventing proximal withdrawal of the access guidewire from the needle shaft.

2. The introducer assembly of claim 1, wherein a proximal portion of the access guidewire has an outer diameter commensurate with the outer diameter of the distal-end portion of the access guidewire, an intermediary portion of the access guidewire between the proximal portion of the access guidewire and the distal-end portion of the access guidewire having an outer diameter less than that of the proximal portion of the access guidewire and the distal-end portion of the access guidewire.

3. The introducer assembly of claim 1, wherein a proximal portion of the distal-end portion of the access guidewire includes a rigid tubular fitting thereover preventing the proximal portion of the distal-end portion of the access guidewire from bending, thereby directing the distal-end portion of the access guidewire to engage the proximal-end portion of the needle slot over a remainder of the needle slot.

4. The introducer assembly of claim 3, wherein the sheath-body opening extends over the proximal-end portion of the needle slot without substantially extending over the remainder of the needle slot such that the sheath body prevents the proximal withdrawal of the access guidewire from the needle shaft by interfering with the distal-end portion of the access guidewire.

5. The introducer assembly of claim 1, wherein the proximal-end portion of the needle slot has a same width as a remainder of the needle slot.

6. The introducer assembly of claim 1, wherein the proximal-end portion of the needle slot is narrower than a remainder of the needle slot.

7. The introducer assembly of claim 1, the introducer sheath further including a splittable sheath hub around the proximal portion of the sheath body.

8. The introducer assembly of claim 7, wherein the sheath hub extends over the remainder of the needle slot exposed by the sheath-body opening such that the sheath hub prevents the proximal withdrawal of the access guidewire from the needle shaft by interfering with the distal-end portion of the access guidewire.

9. The introducer assembly of claim 7, wherein the sheath hub includes a port over the remainder of the needle slot exposed by the sheath-body opening, the port configured to exclusively direct the access guidewire into the proximal-end portion of the needle slot, thereby preventing the proximal withdrawal of the access guidewire from the needle shaft.

10. The introducer assembly of claim 1, further comprising a needle hub around a proximal-end portion of the needle shaft.

11. The introducer assembly of claim 1, further comprising a syringe including a tapered male syringe tip extending from a distal portion of the syringe, the syringe tip configured to insert into a tapered female needle-hub connector in a proximal portion of the needle hub.

12. A rapidly insertable central catheter (“RICC”) insertion assembly, comprising:
a RICC;
an introducer including:
an introducer needle including a needle shaft having a longitudinal needle slot extending from a proximal portion of the needle shaft through a distal needle tip; and
a splittable introducer sheath including a sheath body over the needle shaft sealing the needle slot thereunder but for a sheath-body opening in a proximal portion of the sheath body; and
an access guidewire including a proximal portion disposed in the RICC and a distal-end portion disposed in the introducer, the distal-end portion of the access guidewire having an outer diameter greater than at least a proximal-end portion of the needle slot such that the proximal-end portion of the needle slot catches the distal-end portion of the access guidewire preventing proximal withdrawal of the access guidewire from the needle shaft.

13. The RICC insertion assembly of claim 12, wherein a proximal portion of the access guidewire has an outer diameter commensurate with the outer diameter of the distal-end portion of the access guidewire, an intermediary portion of the access guidewire between the proximal portion of the access guidewire and the distal-end portion of the access guidewire having an outer diameter less than that of the proximal portion of the access guidewire and the distal-end portion of the access guidewire.

14. The RICC insertion assembly of claim 12, wherein a proximal portion of the distal-end portion of the access guidewire includes a rigid tubular fitting thereover preventing the proximal portion of the distal-end portion of the access guidewire from bending, thereby directing the distal-end portion of the access guidewire to engage the proximal-end portion of the needle slot over a remainder of the needle slot.

15. The RICC insertion assembly of claim 14, wherein the sheath-body opening extends over the proximal-end portion of the needle slot without substantially extending over the remainder of the needle slot such that the sheath body prevents the proximal withdrawal of the access guidewire from the needle shaft by interfering with the distal-end portion of the access guidewire.

16. The RICC insertion assembly of claim 12, wherein the proximal-end portion of the needle slot has a same width as a remainder of the needle slot.

17. The RICC insertion assembly of claim 12, wherein the proximal-end portion of the needle slot is narrower than a remainder of the needle slot.

18. The RICC insertion assembly of claim 12, further comprising a splittable sheath hub around the proximal portion of the sheath body.

19. The RICC insertion assembly of claim 18, wherein the sheath hub extends over the remainder of the needle slot exposed by the sheath-body opening such that the sheath hub prevents the proximal withdrawal of the access guidewire from the needle shaft by interfering with the distal-end portion of the access guidewire.

20. The RICC insertion assembly of claim 18, wherein the sheath hub includes a port over the remainder of the needle slot exposed by the sheath-body opening, the port configured to exclusively direct the access guidewire into the proximal-end portion of the needle slot, thereby preventing the proximal withdrawal of the access guidewire from the needle shaft.

21. The RICC insertion assembly of claim 12, further comprising a needle hub around a proximal-end portion of the needle shaft.

22. The RICC insertion assembly of claim 21, further comprising a syringe including a tapered male syringe tip extending from a distal portion of the syringe, the syringe tip configured to insert into a tapered female needle-hub connector in a proximal portion of the needle hub.

23. A method for inserting a rapidly insertable central catheter (“RICC”) into a blood-vessel lumen of a patient, comprising:
acquiring a RICC insertion assembly including the RICC, an introducer, and an access guidewire disposed in both the RICC and the introducer in a substantially ready-to-operate state of the RICC insertion assembly, the introducer including:
an introducer needle including a needle shaft having a longitudinal needle slot extending from a proximal portion of the needle shaft through a distal needle tip; and
a splittable introducer sheath including a sheath body over the needle shaft sealing the needle slot thereunder but for a sheath-body opening in a proximal portion of the sheath body; and
the access guidewire including:
a proximal portion disposed in the RICC; and
a distal-end portion disposed in the introducer, the distal-end portion of the access guidewire having an outer diameter greater than at least a proximal-end portion of the needle slot such that the proximal-end portion of the needle slot catches the distal-end portion of the access guidewire preventing proximal withdrawal of the access guidewire from the needle shaft;
establishing a needle tract from an area of skin to the blood-vessel lumen with the introducer;
advancing a distal end of the access guidewire from its initial location in the introducer proximal of the needle tip into the blood-vessel lumen;
withdrawing the introducer needle from the introducer sheath leaving both the introducer sheath and the access guidewire in place in the blood-vessel lumen, the needle slot allowing the removing of the introducer needle from the introducer sheath while the access guidewire remains in place in the blood-vessel lumen;
splitting the introducer sheath away from the access guidewire to remove split portions of the introducer sheath from the blood-vessel lumen; and
advancing a catheter tube of the RICC over the access guidewire and into the blood-vessel lumen, thereby inserting the RICC into the blood-vessel lumen.

24. The method of claim 23, further comprising:
aspirating blood with a syringe coupled to the introducer needle for confirmation the needle tract extends into the blood-vessel lumen before withdrawing the introducer needle from the introducer sheath.

25. The method of claim 23, further comprising:
withdrawing the access guidewire leaving the catheter tube in place in the blood-vessel lumen.

26. The method of claim 25, further comprising:
advancing a maneuver guidewire into the blood-vessel lumen by way of a primary lumen of the RICC;
advancing a distal portion of the catheter tube farther into the blood-vessel lumen over the maneuver guidewire to a lower ⅓ of a superior vena cava (“SVC”) of a heart of the patient; and
withdrawing the maneuver guidewire leaving the catheter tube in place in the lower ⅓ of the SVC.
